(19) United States
(12) Patent Application Publication
(10) Pub. No.: US 2009/0011035 A1
(43) Pub. Date: Jan. 8, 2009

(54) Personal care composition

(76) Inventors: Joseph Michael Zukowski, Cincinnati, OH (US); Susan Adair Griffiths-Brophy, Middletown, OH (US); Michael Lee Vatter, Okeana, OH (US); Ioannis Constantine Constantinides, Wyoming, OH (US); Chu Zhu, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 11/825,103

(22) Filed: Jul. 3, 2007

(51) Int. Cl. A61K 9/14; A61K 47/06; A61K 31/355; A61P 43/00; A61K 31/455; A61K 47/02

(52) U.S. Cl. 424/498; 514/772; 514/769; 424/490; 514/458; 514/356

(57) ABSTRACT

Stable personal care composition comprising from about 0.1% to about 15% of a non-emulsifying crosslinked siloxane elastomer; from about 0.1% to about 15% of an emulsifying crosslinked siloxane elastomer; from about 1% to about 40% of an elastomer solvent; less than 0.1% of a solidifying agent; an aqueous phase comprising from about 0.0001% to about 10%, by weight of the composition, of a hydrophilic colorant; whereupon application of shear force to the composition a visibly enhanced aqueous phase is visibly separated from the non-aqueous phase.

### FIELD OF THE INVENTION

The present invention relates to personal care compositions, and methods of use thereof, which provide an enhanced visual indication of water release from the composition upon application.

### BACKGROUND OF THE INVENTION

A variety of products are available to the consumer to provide skin care benefits and to counteract what many consider undesirable “signs of skin aging,” such as fine lines, wrinkles and uneven skin texture. To be most effective, some products must be applied regularly and over an extended period of time. This may be especially important when the product is intended to provide a chronic, or long-term, benefit. To encourage frequent usage, it is important that the product have a desirable feel when applied, and also provides some indication that the product is having its intended effect. There exists a continuing need, therefore, to provide personal care compositions that signal an immediate benefit and thus encourage repeated use to provide a long-term benefit.

One example of an immediate benefit occurs in compositions that release an aqueous phase upon application of shear force, for example, by applying the composition the skin. Such compositions provide a water-like, fresh feel upon application, and leave the consumer with a silky after-feel. When the release of water is visibly discernable by the consumer, enjoyment and motivation to use the product is increased. Often, however, the water release is not readily apparent, in particular when the composition and/or the water are relatively colorless, or are similar in color. Color may be imparted by the addition of colorants such as dyes and pigments. The choice of colorants, however, may be limited by incompatibility with certain actives and/or may result in a composition that has an undesirable stability, appearance or feel. For example, some particulate materials are unsuitable for use in emulsions, and result in agglomeration of the particulates (i.e. flocculation or precipitation), clumping, graininess, and/or separation of the aqueous and non-aqueous phases, all of which result in compositions that have an unpleasant appearance and/or feel. In addition, the colorant must selectively partition into the aqueous phase to increase visibility. There exists a need, therefore, to provide stable skin care compositions which provide an immediate benefit in the form of a visibly enhanced water release, are stable and which have a pleasant feel upon application.

### SUMMARY OF THE INVENTION

The present invention meets the aforementioned needs, and describes stable compositions and methods of use thereof in which the aqueous phase is separated from the non-aqueous phase, or coalesces, when the composition is applied to keratinous tissue. Applicants have found that hydrophilically coated particulate materials can be incorporated into the aqueous phase to visibly enhance the separated aqueous phase. In addition, Applicants have found that water-soluble colorants and dyes can be selectively incorporated into the aqueous phase. The resulting compositions have a pleasant appearance, feel and stable physical properties and also a visibly enhanced aqueous phase upon separation from the non-aqueous phase. In addition to color, the appearance of the aqueous phase also may be enhanced, for example, by a difference between the phases in opacity, reflectivity, transmittance, and/or other optical properties.

The following represent some non-limiting embodiments of the present invention.

According to the first embodiment of the present invention, a stable personal care composition is provided comprising from about 0.1% to about 15% of a non-emulsifying crosslinked siloxane elastomer; from about 0.1% to about 15% of an emulsifying crosslinked siloxane elastomer; from about 1% to about 40% of an elastomer solvent; less than 0.1% of a solidifying agent; an aqueous phase comprising from about 0.0001% to about 10%, by weight of the composition, of a hydrophilic colorant; whereupon application of shear force to the composition a visibly enhanced aqueous phase is visibly separated from the non-aqueous phase.

According to a second embodiment of the present invention, a stable personal care composition is provided comprising from about 0.1% to about 15% of an emulsifying silicone elastomer; from about 0.1% to about 40% of at least one solidifying agent; from about 1% to about 75% of an aqueous phase; from about 0.1% to about 74% water; from about 0.1% to about 10% of a hydrophilically-coated particulate comprising a core material and a coating material; wherein the composition is in the form of a water-in-oil emulsion, has a first hardness of from about 2 g to about 45 g at a first temperature of 21° C., and a second hardness at a second temperature of 33° C., wherein the second hardness is 65% or less of the first hardness; and whereupon application of shear force to the composition a visibly enhanced aqueous phase is visibly separated from the non-aqueous phase.

According to another embodiment of the present invention, a method of providing a benefit to mammalian keratinous tissue is provided, comprising the step of applying a composition to mammalian keratinous tissue according to the first and/or second embodiment of the present invention. Alternatively, the present invention provides a method of signaling a short-term benefit to a consumer, comprising the step of applying a composition to mammalian keratinous tissue according to the first and/or second embodiment of the present invention.

According to yet another embodiment of the present invention, a kit is provided, comprising the composition according to the first and/or second embodiment of the present invention.

### DETAILED DESCRIPTION OF THE INVENTION

The present invention describes compositions which signal an immediate benefit to a consumer by releasing a visually enhanced aqueous phase upon application to mammalian keratinous tissue, and methods of use thereof. The composition may be used in a variety of personal care products, non-limiting examples of which include moisturizers, skin conditioners, skin cleansers, sunscreens, anti-aging compounds, and combinations thereof. The composition may be in a variety of forms, including but not limited to, a lotion, a solid, a cream, a gel, a mousse, an ointment, a paste, a serum, a stick, etc.

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Herein, “personal care composition” means compositions suitable for topical application on mammalian keratinous tissue. “Skin care actives,” or “actives,” as used herein, means compounds that, when applied to the skin, provide a benefit or improvement to the skin. It is to be understood that skin care actives are useful not only for application to skin, but also to hair, nails and other mammalian keratinous tissue.

Herein, “stable” and “stability” mean a composition which is substantially unaltered in chemical state, physical homogeneity and/or color, for example, the composition does not exhibit undue flocculation, precipitation and/or phase separation, upon exposure to conditions reasonably expected to be incurred in shipping, storage and use. Stability may be determined either by empirical observation or by appropriate methods of chemical and/or physical analysis that would be known to one of skill in the art to determine such properties as viscosity, particle size or rheology.

“Keratinous tissue,” as used herein, refers to keratin-containing layers disposed as the outermost protective covering of mammals which includes, but is not limited to, skin, hair, nails, cuticles, etc.

“Dermatologically acceptable,” as used herein, means that the compositions or components described are suitable for use in contact with human keratinous tissue without undue toxicity, incompatibility, instability, allergic response, and the like.

“Immediate,” as used herein, means that the benefit occurs when the composition is applied to the keratinous tissue as described herein.

“Visibly separated,” as used herein, means that a composition in the form of an emulsion releases the aqueous phase (i.e. is “water-releasing”) upon application to the skin or other keratinous tissue. Whether a composition is water-releasing may be determined by microscope-assisted visual analysis of an emulsion having an average water droplet size of about 3 microns (μm) or less. It should be understood that if a sample exhibits an average water droplet size of greater than 3 microns, the sample may not be properly characterized by microscopy; however, the sample may be characterized by the rheological method, also described herein. A standard optical microscope with Differential Interference Contrast and Crossed Polarized Light capabilities and an optical shear stage is used. Cross polarization may be used for sample compositions that have low translucency or for characterization of the watery domains. With the cross polarization technique, watery domains will appear dark in the resulting image. A suitable configuration includes a Zeiss Axioplan™ 2 microscope (available from Carl Zeiss, Inc, Thornwood, N. Y.) coupled with a MTI 3CCD camera (available from DAGE-MTI™, Michigan City, Ind.). Images are acquired using Metamorph™ software version 6.1 (available from Molecular Devices Corporation, Sunnyvale, Calif.), which is used to measure droplet size and save the resulting image. The microscope is paired with a CSS450 optical shear stage (available from Linkam Scientific Instruments, Surrey, UK) and is configured to provide 500× magnification. About 1.5 g of the emulsion (“sample”) is carefully loaded onto the shear stage to minimize shear. The shear system is configured for a steady mode having a gap width of 1 mm and a constant shear rate of 16 s⁻¹. Temperature is held constant at approximately 25° C. An initial micrograph is captured of the sample prior to initiation of shear by the shear stage. The sample is subjected to 15 seconds of shear, the shear is discontinued, and a micrograph is captured. This is repeated 5 times (e.g., sample is subjected to a cumulative 90 seconds of shear) to yield five micrographs (e.g., taken at time =0, 15, 30, 45, 60, 75, and 90 seconds). The visible water domains of the sample are analyzed to provide a maximum linear dimension for each of the visible water domains. Three samples of each emulsion are tested. A visibly separated aqueous phase occurs when an amorphous aqueous region having a maximum linear dimension of at least about 10 microns becomes visible at 500× magnification within about 1 minute of shear. In alternate embodiments, the visibly separated aqueous phase occurs when an amorphous region of water having a size of at least about 25, 50, or 75 microns becomes visible at 500× magnification within about 1 minute of shear. In another suitable embodiment, the visibly separated aqueous phase occurs when an amorphous region of water having a size of at least 10 microns becomes visible at 500× magnification within about 45 second, 30 second, or 15 seconds of shear. Compositions that do not release when applied to the skin do not exhibit a significant change in the water droplet size when exposed to these conditions.

For determination of whether an emulsion having an average water droplet size of greater than 3 microns is “water-releasing,” a rheological profile for an emulsion (“sample”) may be obtained as follows. The sample is evaluated using an AR 2000 Rheometer available from TA Instruments™, New Castle, Del. that is interfaced with a computer having software that provides data recordation and analysis. The rheometer is configured with 4 cm flat plates at a gap setting of 1000 microns, a temperature of 25° C., and in a controlled stress mode. The rheometer is configured to ramp stress from 1 Pa to 1000 Pa with a duration of 3 minutes and to sample at a rate of 10 points per decade. A rheology profile is plotted using the log₁₀ viscosity (Pa·s) on the y-axis versus the log₁₀ shear stress (Pa) on the x-axis. Water-releasing compositions exhibit a sharp decrease in viscosity at a critical shear stress. This decrease in viscosity may be measured as the slope of the plot between the regions wherein the viscosity has a substantially constant high viscosity and a substantially constant lower viscosity. The slope is calculated according to the formula [(log viscosity(t2)−log viscosity(t1)]/[(log shear stress(t2)−log shear stress (t1)], where viscosity (t1) and viscosity (t2) are the viscosity readings before and after the viscosity value decreases 10 fold (which on the log scale is a change of 1.0) between two readings, and the shear stress (t1) and shear stress (t2) are the corresponding shear stress readings. If the viscosity decreases gradually and no sudden viscosity drop of more than 10 fold between two readings occurs, any representative readings on the plot can be used for the slope calculation. For water-releasing compositions, the slope of the region of the plot exhibiting a sharp decrease is less than about −5. In alternate embodiments, slope of the region of the plot exhibiting a sharp decrease is less than about −10, −25, −50, −75, or −100.

“Visible enhancement,” or “visibly enhanced,” as used herein, means that the aqueous phase isolated as described above has a percent transmittance (% T) of less than 65% of the % T of a pure water control sample, determined as described herein. Percent transmittance may be quantified by measuring the transmittance of the aqueous phase after separation from the non-aqueous phase. The aqueous phase may be separated from the emulsion by exposing the emulsion to high shear by the use of mills and/or ultrasonic probes. In addition, separating the aqueous phase from the non-aqueous phase can be facilitated by addition of a water miscible solvent such as pentylene glycol. For example, dilute 13 grams of a water in silicone emulsion with 1.6 g of pentylene glycol. Using a hand held homogenizer (IKA T8 100W, 2500 rpm), mill the sample for approximately 2.5 minutes. Decant the aqueous phase. Centrifuge the decanted water at 14,000 rpm for 3 minutes using an Eppendorf Centrifuge 5415C. Remove any solids from the surface of the water. Shake the remaining solution to resuspend any remaining pigments and obtain a transmittance spectrum from 400 nm to 800 nm using a Hewlett Packard Model 8453 spectrophotometer. The percent transmittance (% T) of an aqueous phase acquired from a non-visibly enhanced water-in-silicone emulsion (e.g. no dyes or pigments) is 65% or greater than the % T of a distilled water control sample. For visibly enhanced water release products, the aqueous phase isolated as described above has a % T of less than 65% of the % T of a pure water control sample.

“Hardness,” as used herein, means the amount of force in grams (g) necessary for a probe having a diameter of 2 mm to penetrate a distance of 0.3 mm at a rate of 0.1 mm/s into the composition, using a TA-XT2i Texture Analyzer with Texture Expert Exceed software (v. 2.64). Prior to measuring the hardness, the composition is allowed to equilibrate to a first temperature or a second temperature, as indicated herein. When no temperature is indicated, the temperature of the composition is 25° C. The first and/or second temperature is understood to be ±1° C.

Herein, “delivery enhancement device” means any device that increases the amount of active ingredient applied to and/or into the skin relative to the amount of active ingredient that is delivered without using the device.

Herein, “regulating skin condition” means improving skin appearance and/or feel, for example, by providing a smoother appearance and/or feel. Herein, “improving skin condition” means effecting a visually and/or tactilely perceptible positive change in skin appearance and feel. Conditions that may be regulated and/or improved include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

As used herein, “signs of skin aging,” include, but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

### I. Composition

The composition of the present invention comprises a non-aqueous phase and an aqueous phase, and is in the form of an emulsion. Suitable types of emulsions include, but are not limited to, oil-in-water, water-in-oil, water-in-oil-in-water, and oil-in-water-in-oil emulsions. The oil may be derived from animals, plants, or petroleum, may be natural or synthetic, and may comprise silicone oils. In one embodiment, the dermatologically acceptable carrier comprises oil-in-water emulsions and water-in-oil emulsions, and alternatively is a water-in-oil emulsion.

Upon application to the keratinous tissue by shear force, the aqueous phase becomes visibly separated from the non-aqueous phase. The non-aqueous phase typically is substantially evenly distributed upon the skin. The aqueous phase may become visibly separated immediately upon application, and alternatively within about three to about five seconds.

The visibly separated aqueous phase is visibly distinct from the non-aqueous phase. For example, the aqueous phase may have a different color than the non-aqueous phase. In one embodiment, the non-aqueous phase is substantially colorless, white or colorless, and the aqueous phase is colored. Alternatively, the aqueous phase may comprise particulate materials. The particulate materials may reflect and/or refract one or more colors that are visibly distinct from the non-aqueous phase. Alternatively, the particulate materials may reflect white light (or “sparkle”). Alternatively, the aqueous phase may be opaque relative to the non-aqueous phase, for example wherein the non-aqueous phase is clear and/or colorless and wherein the aqueous phase is opaque.

Examples of shear force include applying to the skin, or other keratinous tissue, for example by lightly smearing, rubbing, dabbing, wiping, etc. with a finger, hand, implement and/or a delivery enhancement device. The separate aqueous phase may provide immediate benefits, including but not limited to, an immediate indication that the product is having its intended effect, an indication of the presence of one or more skin care actives, and/or an enhanced pleasant, “silky” feel upon application. After separation of the phases, the aqueous phase may be rubbed into the skin, may be allowed to evaporate, or otherwise manipulated.

When the composition comprises at least 0.1% of a solidifying agent, the composition of the present invention may have a first hardness, measured as described herein, at a first temperature, and a second hardness, also measured as described herein, at a second temperature. The first hardness may be from about 2 g to about 45 g, alternatively from about 2 g to about 40 g, alternatively from about 5 g to about 35 g, alternatively from about 5 g to about 20 g, and alternatively from about 5 g to about 12 g, at a first temperature of 21° C. The second hardness, at a second temperature of 33° C., may be about 65% or less, alternatively about 55% or less, alternatively about 45% or less, and alternatively about 30% or less, of the first hardness. Alternatively, the first hardness of the composition at a first temperature of 21° C. decreases by at least 35%, and alternatively by at least 45%, alternatively by at least 55%, and alternatively by at least 70%, at a second temperature of 33° C. Alternatively, the second hardness at a second temperature of 33° C. is from about 0.1 g to about 30 g, alternatively from about 0.1 g to about 20 g, and alternatively from about 0.1 g to about 10 g, and alternatively from about 0.4 g to about 5 g.

The composition of the present invention may have a spreadability, measured as described herein, 1,000 g·s to about 10,000 g·s at a temperature of 21° C. of from about of from about 500 g·s to about 2,500 g·s at a temperature of 33° C. In one embodiment, the spreadability of the composition at 33° C. is from about 10% to about 50%, and alternatively from about 20% to about 35%, of the spreadability of the composition at 21° C.

The composition of the present invention is stable as defined herein when the composition is at a temperature of about 40° C. In one embodiment, a composition is described which exhibits signs of instability at a temperature of above 40° C. and which is fit for the originally intended use when the composition is cooled to a temperature of about 40° C. or less. For example, if the composition melts and is again cooled, the composition substantially resumes its stable form and retains desirable properties such as skin feel and appearance, and is suitable for use as described herein.

The composition may maintain rheology when hardness is reduced as described herein, at an elevated temperature. The stability of the rheology may be measured after the composition has equilibrated to a substantially uniform temperature of 45° C.±1° C. with a Brookfield™ RVDV-II+Viscometer on a Brookfield Helipath Stand equipped with a T-bar spindle (size C) rotating at 5 rpm. The viscosity may be measured at one or more points as the spindle is moved in a downward direction through previously undisturbed product. The composition may have a viscosity of from about 5,000 centipoise (cps) to about 500,000 cps, alternatively from about 10,000 cps to about 300,000 cps, alternatively from about 20,000 cps to about 200,000 cps, and alternatively from about 40,000 cps to about 140,000 cps, all at 45° C.

### A. Non-Aqueous Phase

The composition may comprise from about 1.2% to about 70.1% of a non-aqueous phase. In an alternative embodiment, the composition may comprise from about 0.1% to about 65% of a non-aqueous phase. The non-aqueous phase may comprise an emulsifying and/or non-emulsifying silicone elastomer, one or more oil-soluble skin care actives, one or more solidifying agents, an elastomer solvent, and mixtures thereof.

### 1. Elastomers

The composition of the present invention comprises a silicone elastomer, useful for reducing the tackiness of the composition and for providing a pleasant feel upon application. One example of useful silicone elastomers are crosslinked organopolysiloxane (or siloxane) elastomers, as described in U.S. patent publication 2003/0049212A1. When the composition is in the form of an emulsion, the composition may comprise emulsifying and non-emulsifying silicone elastomers. “Emulsifying,” as used herein, means crosslinked organopolysiloxane elastomers having at least one polyoxyalkylene (e.g., polyoxyethylene or polyoxypropylene) or polyglycerin moiety, whereas “non-emulsifying” means crosslinked organopolysiloxane elastomers essentially free of polyoxyalkylene or polyglycerin moeities.

The composition of the present invention may comprise from about 0.1% to about 15%, alternatively from about 0.1% to about 5%, and alternatively from about 0.1% to about 2% of a non-emulsifying crosslinked siloxane elastomer. In one embodiment, the non-emulsifying crosslinked siloxane elastomers are dimethicone/vinyl dimethicone crosspolymers, supplied by a variety of suppliers including Dow Corning™ (DC 9040 and DC 9041), General Electric™ (SFE 839), Shin Etsu™ (KSG-15, 16, 18 [dimethicone/phenyl vinyl dimethicone crosspolymer]), and Grant Industries (GRANSIL™ line of elastomers). Cross-linked siloxane elastomers useful in the present invention and processes for making them are further described in U.S. Pat. No. 4,970,252 to Sakuta, et al.; U.S. Pat. No. 5,760,116 to Kilgour, et al.; and U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo KK. In addition, suitable organopolysiloxane elastomer powders include vinyl dimethicone/methicone silesquioxane crosspolymers such as KSP-100, KSP-101, KSP-102, KSP-103, KSP-104, KSP-105 (Shin Etsu™); hybrid silicone powders comprising a fluoroalkyl group, such as KSP-200 (Shin Etsu™); and hybrid silicone powders comprising a phenyl group, such as KSP-300 (Shin Etsu™) and DC-9506 (Dow Corning™).

The composition of the present invention may comprise from about 0.1% to about 15%, alternatively from about 0.2% to about 5%, and alternatively from about 0.2% to about 2% of an emulsifying crosslinked organopolysiloxane elastomer, described in U.S. Pat. Nos. 5,412,004; 5,837,793; and 5,811,487. Non-limiting examples of suitable emulsifying elastomers include polyoxyalkylene-modified elastomers formed from divinyl compounds, e.g. siloxane polymers with at least two free vinyl groups bonded via Si—H linkages on a polysiloxane backbone. In one embodiment, the emulsifying crosslinked organopolysiloxane elastomers are dimethyl polysiloxanes crosslinked by Si—H sites on a molecularly spherical MQ resin (R3SiO_{1/2}SiO_{4/2}), and alternatively is dimethicone copolyol crosspolymer and dimethicone, commercially available from Shin Etsu as KSG-21.

### 2. Elastomer Solvent

The composition of the present invention may comprise from about 1% to about 40%, alternatively from about 4% to about 40%, and alternatively from about 5% to about 40% of a suitable solvent for the crosslinked organopolysiloxane elastomers, described in U.S. patent publication 2003/0049212A1. The concentration of the solvent in the cosmetic compositions of the present invention may vary depending upon the type and amount of solvent and the cross-linked siloxane elastomer employed, and when combined with the cross-linked organopolysiloxane elastomer particles of the present invention, serves to suspend and swell the elastomer particles to provide an elastic, gel-like network or matrix. The carrier for the cross-linked siloxane elastomer is liquid under ambient conditions, and preferably has a low viscosity to provide for improved spreading on the skin.

The solvent may comprise volatile, non-polar oils; non-volatile, relatively polar oils; non-volatile, non-polar oils; and non-volatile paraffinic hydrocarbon oils. The term “non-volatile” as used herein refers to materials that exhibit a vapor pressure of no more than about 0.2 mm Hg at 25° C. at one atmosphere and/or to materials that have a boiling point at one atmosphere of at least about 300° C. The term “volatile” as used herein refers to all materials that are not “non-volatile” as previously defined herein. The phrase “relatively polar” as used herein means more polar than another material in terms of solubility parameter; i.e., the higher the solubility parameter the more polar the liquid. The term “non-polar” typically means that the material has a solubility parameter below about 6.5 (cal/cm³)^{0.5}.

Non-limiting examples of suitable non-polar, volatile oil are disclosed in U.S. Pat. No. 4,781,917 issued to Luebbe et al. and include polydecanes such as isododecane and isodecane (e.g., Permethyl-99A, available from Presperse™ Inc.) and C7-C15 isoparaffins (e.g. the Isopar Series, from Exxon™ Chemicals); cyclomethicones of varying viscosities, e.g., Dow Corning™ 200, Dow Corning™ 244, Dow Corning™ 245, Dow Corning™ 344, and Dow Corning™ 345, Silicone Fluids, commercially available from G.E. Silicones, (e.g. SF-1204, SF-1202, GE 7207 and GE 7158); and SWS-03314 (commercially available from SWS Silicones™ Corp.).

Relatively polar, non-volatile oils useful in the present invention include, but are not limited to, silicone oils; hydrocarbon oils; fatty alcohols; fatty acids; esters of mono and dibasic carboxylic acids with mono and polyhydric alcohols; polyoxyethylenes, polyoxypropylenes, mixtures of polyoxyethylene and polyoxypropylene ethers of fatty alcohols; and mixtures thereof. In one embodiment, the polar, non-volatile oil is selected from the group consisting of propoxylated ethers of C14-C18 fatty alcohols having a degree of propoxylation below about 50, esters of C2-C8 alcohols and C12-C26 carboxylic acids (e.g. ethyl myristate, isopropyl palmitate), esters of C12-C26 alcohols and benzoic acid (e.g. Finsolv™ TN supplied by Finetex™), diesters of C2-C8 alcohols and adipic, sebacic, and phthalic acids (e.g., diisopropyl sebacate, diisopropyl adipate, di-n-butyl phthalate), polyhydric alcohol esters of C6-C26 carboxylic acids (e.g., propylene glycol dicaprate/dicaprylate, propylene glycol isostearate); and mixtures thereof.

Examples of suitable non-volatile, non-polar oils include, but are not limited to non-volatile polysiloxanes, paraffinic hydrocarbon oils, and mixtures thereof. The polysiloxanes useful in the present invention selected from the group consisting of polyalkylsiloxanes, polyarylsiloxanes, polyalkylarylsiloxanes, poly-ethersiloxane copolymers, and mixtures thereof. Examples of useful oils include Viscasil™ series (General Electric); the Dow Corning 200 series (Dow Corning Corp.); SF 1075 methyl-phenyl fluid (General Electric) and 556 Cosmetic Grade Fluid (Dow Corning Corp.).

Non-volatile paraffinic hydrocarbon oils useful in the present invention are described in U.S. Pat. No. 5,019,375 issued to Tanner et al. and in 2003/0049212A1, and include mineral oils and branched-chain hydrocarbons such as Permethyl™ 102A, 103A and 104A (Permethyl Corporation); and Ethylflo™ 364 (Ethyl Corp.). Additional suitable solvents useful herein are described in U.S. Pat. No. 5,750,096 to Guskey et al.

### 3. Solidifying Agent

The composition of the present invention may comprise one or more solidifying agents suitable to impart stability to the composition at a temperature of about 40° C. and to impart a suitable hardness as described herein. In one embodiment, the composition may comprise less than 0.1% of a solidifying agent. In an alternative embodiment, the composition may comprise from about 0.1% to about 40%, alternatively from about 1% to about 30%, and alternatively from about 5% to about 25%, of a solidifying agent. A variety of solidifying agents may be used, including those disclosed in U.S. Pat. No. 6,696,049, issued to Vatter et al. In one embodiment, the solidifying agent is a wax.

Waxes suitable for use herein include but are not limited to animal, vegetable, mineral, or silicone waxes. Generally such waxes have a melting point ranging from about 25° C. to 125° C., and alternatively from about 30° C. to about 100° C. Non-limiting examples of suitable waxes include silicone waxes, fatty esters, for example cetyl and/or stearyl esters, acacia, beeswax, ceresin, flower wax, citrus wax, carnauba wax, jojoba wax, japan wax, polyethylene, synthetic waxes, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, polyalkylene glycol derivatives thereof (for example PEG6-20 beeswax, or PEG-12 carnauba wax) and mixtures of any of the aforementioned waxes. In one embodiment, the wax is a polyethylene wax, and alternatively is a polyethylene wax having a melting point of less than 120° C., alternatively less than 95C, and alternatively less than 85° C.

Non-limiting examples of suitable silicone waxes are disclosed in U.S. Pat. Nos. 5,413,781 and 5,725,845, and further include alkylmethyl polysiloxanes, C10-C60 alkyl dimethicones, and mixtures thereof. Alternatively, the silicone wax may be a C16-C28 alkyl dimethicone wax. Other suitable silicone waxes include, but are not limited to stearoxydimethicone, behenoxy dimethicone, stearyl dimethicone, cetearyl dimethicone, cetyl dimethicone, and mixtures thereof.

### B. Aqueous Phase

The composition of the present invention comprises an aqueous phase. In one embodiment the composition comprises from about 30% to about 98.8%, alternatively from about 40% to about 95%, and alternatively from about 65% to about 90% of the aqueous phase. In an alternative embodiment, the composition comprises from about 0.1% to about 75%, alternatively from about 1% to about 70%, and alternatively from about 10% to about 50%, of the aqueous phase. The aqueous phase may in turn comprise an additional emulsifier, a hydrophilic colorant, one or more water-soluble skin care actives, and mixtures thereof.

### 1. Additional Emulsifier

The composition of the present invention may contain an additional emulsifier, useful for dispersing and suspending the aqueous phase within the non-aqueous phase in a water-in-oil emulsion. If present, the composition may comprise from about 0.001% to about 5%, alternatively from about 0.01% to about 5% alternatively from about 0.1% to about 3%, and alternatively from about 0.1% to about 2%, of at least one additional emulsifier.

A wide variety of emulsifying agents can be employed herein to form a water-in-silicone emulsion, and are described in U.S. patent publication 2003/0049212A1. In one embodiment, the additional emulsifiers are silicone emulsifiers, including organically modified organopolysiloxanes (silicone surfactants) such as dimethicone copolyols. Examples of commercially available dimethicone copolyols useful herein are Dow Corning® 190, 193, Q2-5220, 2501 Wax, 2-5324 fluid, and 3225C; ABIL™ EM-90, ABIL™ WE-09 and ABIL® WS-08 (Goldschmidt), KF-6028 and KF-6106 (Shin-Etsu™).

In one embodiment, the additional emulsifier is a non-silicone emulsifier, non-limiting examples of which include non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof.

### 2. Colorants

The composition of the present invention comprises a colorant. In one embodiment the colorant is a hydrophilic colorant and is present in the aqueous phase. The aqueous phase may comprise from about 0.0001% to about 10%, alternatively from about 0.0005% to about 10%, alternatively from about 0.1% to about 5%, and alternatively from about 0.2% to about 2% by weight of the composition, of a colorant. Suitable classes of colorants include, but are not limited to, dyes, toners, pigments, and particulate materials, including hydrophilic and/or hydrophilically-coated particulates.

Nonlimiting examples of particulate materials useful in the present invention include colored pigments, pigments which themselves are uncolored but which reflect or transmit one or more colors (such as interference pigments), inorganic powders, organic powders, composite powders, optical brightener particles, exfoliants, and combinations thereof. Also useful are lakes, primary FD&C and D&C lakes and blend thereof. These particulates can be platelet shaped, spherical, elongated or needle-shaped, or irregularly shaped, surface coated or uncoated, porous or non-porous, charged or uncharged, and can be added to the current compositions as a powder or as a pre-dispersion. In one embodiment, the particulate material has an average primary particle size of from about 0.1 microns to about 100 microns, more preferably from about 0.1 microns to about 75 microns, and still more preferably from about 0.1 microns to about 25 microns. In an alternative embodiment, the particulate material has an average primary particle size of from about 5 microns to about 100 microns, alternatively from about 5 microns to about 75 microns, alternatively from about 5 microns to about 25 microns, alternatively from about 0.1 microns to about 0.3 microns, and alternatively from about 0.3 microns to about 1 micron.

Non-limiting examples of particulate materials useful herein include bismuth oxychloride, iron oxide, mica, mica treated with barium sulfate or other materials, titanium dioxide (TiO2), zinc oxide, zirconium oxide, silica, nylon, polyethylene, talc, styrene, polypropylene, ethylene/acrylic acid copolymer, sericite, aluminum oxide, silicone resin, barium sulfate, calcium carbonte, cellulose acetate, polymethyl methacrylate, and mixtures thereof. Examples of commercially available inorganic particulate materials include the TRONOX™ TiO2 series, SAT-T CR837, a rutile TiO2 (U.S. Cosmetics), and COVERLEAF™ AR-80, which comprises layered inorganic compounds (Catalysts and Chemicals Ind. Co. Ltd.), and Goddbal™, a silica powder encapsulating one or more inorganic chemicals (Suzuki Yushi Ind. Co. Ltd.). In one embodiment, the particulate material is a charged dispersion of titanium dioxide, as disclosed in U.S. Pat. No. 5,997,887.

The composition further may comprise organic powders and/or fillers, including but not limited to, polymeric particles such as methylsilsesquioxane resin microspheres (for example Tospearl™ 145A, available from Toshiba), microspheres of polymethylmethacrylates (for example Micropearl™ M 1001d, available from Seppic); the spherical particles of crosslinked polydimethylsiloxanes, (for example Trefil™ E 506C or Trefil™ E 505C available from Dow Corning™ Toray Silicone); sphericle particles of polyamide (for example Nylon 12, especially such as those available from Atochem™ under the name Orgasol™ 2002D Nat C05); polystyerene microspheres (for example Dyno Particles™ sold under the name Dynospheres™); ethylene acrylate copolymer (for example FloBead™ EA209, available from Kobo), and mixtures thereof.

The composition of the present invention may comprise interference pigments, for the purpose of the present invention defined as thin platelike layered particles having two or more layers of controlled thickness with different refractive indices. The layers yield a characteristic reflected color from the interference of typically two, but occasionally more, light reflections, from different layers of the platelike particle. The interference pigment may comprise mica layered with about 50 nm to about 300 nm films of TiO2, Fe2O3, silica, tin oxide, and/or Cr2O3. Such pigments often are pearlescent. Non-limiting examples of commercially available interference pigments include Timiron™ and Dichrona (Rona); Prestige and Prestige Silk™ (Eckart); and Sicopearls™ (BASF). In one embodiment, the interference pigments have an average diameter of individual particles less than about 75 microns in the longest direction, preferably with an average diameter less than about 50 microns.

The hydrophilic colorant may comprise a particulate comprising a core material, or uncoated particulate, and a coating material which at least partially covers the core material (“coated particulate”). In one embodiment, the coating material is hydrophilic. The coated particulate may have an average primary particle size of from about 0.1 microns to about 100 microns, more preferably from about 0.1 microns to about 75 microns, and still more preferably from about 0.1 microns to about 25 microns. In an alternative embodiment, the particulate material has an average primary particle size of from about 5 microns to about 100 microns, alternatively from about 5 microns to about 75 microns, alternatively from about 5 microns to about 25 microns, alternatively from about 0.1 microns to about 0.3 microns, and alternatively from about 0.3 microns to about 1 micron.

The coated particulate may be present in an amount of from about 0.1% to about 10%, more preferably from about 0.1% to about 5%, more preferably from about 0.2% to about 2%, and still more preferably from about 0.2% to about 1%. The core material may comprise essentially any material capable of being at least partially coated with one or more coating materials described herein. In one embodiment, the core material is selected from the group consisting of mica, iron oxide, titanium dioxide, boron nitride, interference pigments, and mixtures thereof, alternatively is an interference pigment, and alternatively is mica.

The coating material may comprise water-soluble amino acids, collagen, lecithin, polyacrylate/lecithin, chitosan, and mixtures thereof. The coating material further may comprise combinations of one or more anchoring groups (A) and stabilizing groups (S). The anchoring group is attached to the core material, and the stabilizing group is in contact with and/or interacts with the carrier solvents. The stabilizing group may serve to modify the surface properties of the coated particulate, such as surface energy, polarity, and solubility. Additionally, both anchoring groups and stabilizing groups may be attached to other anchoring and/or stabilizing groups to produce a variety of coating materials. In any combination, however, the core material will be bonded to at least one anchoring group, and at least one stabilizing group will be in contact with and/or interact with the carrier solvent(s). The bond between the anchoring group and the core material may be covalent or non-covalent (adsorbed), but is substantially irreversible, understood herein to mean that the coating material remains bonded to the core material under normal mixing, storage and use conditions. Specifically, in the event multiple anchoring groups are bonded to the core material, one or more anchoring groups may reversibly bind to the core material; however, a sufficient amount of anchoring groups will remain bonded so that the coating material on the whole is substantially irreversibly bonded. In one embodiment, the bond between the anchoring group and the core material is covalent. When adsorbed to the core material in a solution or in a composition described herein, the coating material may form a layer having a thickness of at least 10 nanometers (>10 nm), and preferably from about 10 nm to about 100 nm. Non-limiting examples of coating materials comprising anchoring groups and stabilizing groups which are suitable for use herein include Dow Corning polymers 208822A, 2-8040, 8566, AP6087, and mixtures thereof.

In one embodiment, the anchoring group is a silanol group and the stabilizing group is a polar moiety. Non-limiting examples of suitable polar moieties include polyethyleneoxide, polyethylene glycols, polypropylene glycol, polyethylene glycol alkyl, alkyl glycols, alkyl glycol ether, polyethylene glycol esters, polyalkylene oxide, polypropyleneoxides, polyglycerides and mixtures thereof. In an alternative embodiment, the anchoring group is selected from the group consisting of carboxyls, carboxylic acid esters, hydroxyls, sulfonates, sulfates, phosphates, phosphonates, nitros, carbohydrates, ammonium salts, phosphate esters, carbonyls, aminos, amides, imides, aliphatic hydrocarbons, aromatic hydrocarbons, heterocyclic groups, polypropyleneoxides, silicones, fluorocarbons, polyesters, urethanes and mixtures thereof, and wherein the stabilizing group is selected from the group consisting of polyethyleneoxide, polyethylene glycols, polypropylene glycol, polyethylene glycol alkyl, alkyl glycols, alkyl glycol ether, polyethylene glycol esters, polyalkylene oxide, polypropyleneoxides, polyglycerides, carboxyl, carboxylic acid esters, hydroxyl, sulfonate, sulfate, phosphate, phosphonate, nitro, carbohydrate, ammonium salts, phosphate esters, carbonyl, amino, amide, imide, aliphatic hydrocarbons, aromatic hydrocarbons, heterocyclic groups, polypropyleneoxides, silicones, fluorocarbons, polyesters and mixtures thereof.

The coated particulate may have a surface energy comprising a polar component and a dispersive component. In one embodiment, the polar component is greater than 20 mJ/m² and the dispersive component is greater than 15 mJ/m², and alternatively the polar component is from about 20 mJ/m² to about 55 mJ/m² and the dispersive component is from about 15 mJ/m² to about 65 mJ/m², where “mJ” means 1×10⁻³ Joules and “m²” means square meters. The total surface energy of the hydrophilic colorant is the sum of the dispersive surface energy component and the polar surface energy component. The surface energy of the hydrophilic colorant is derived from contact angle measurements, which can be converted to surface energy by various accepted models that would be known to one of skill in the art. One such model, used in the present invention, is the Fowkes equation, as described in Fowkes, F. M.: Industrial and Engineering Chemistry, vol. 56, number 12, p. 40 (1964):

γyₗᵥ(1+cos θ)=2(γₗᵥ^{d}γₛᵥ^{d}) 1/2+2(γₗᵥ^{p}γₛᵥ^{p}) 1/2

where θ refers to the contact angle; γₗᵥ refers to the surface tension of liquid; γₗᵥ^{d} refers to is the dispersive component of the surface tension of liquid; γₛᵥ^{d} refers to the dispersive component of the surface tension of solid; γₗᵥ^{p} refers to the polar component of the surface tension of liquid and γₛᵥ^{p}—is the polar component of the surface tension of solid. The contact angles of the coating material were measured using diiodomethane (99%, Aldrich), ethylene glycol (99%+, Aldrich) and water (HPLC grade, Aldrich).

In one embodiment, the composition may be substantially free from titanium dioxide, wherein “substantially free” is understood to include less than 0.001%.

### C. Actives

The compositions of the present invention may comprise at least one additional skin care active, useful for regulating and/or improving the condition of mammalian skin. Classes of suitable skin care actives include, but are not limited to vitamins, peptides and peptide derivatives, sugar amines, sunscreens, oil control agents, flavonoid compounds, hair growth regulators, antioxidants and/or antioxidant precursors, preservatives, phytosterols, protease inhibitors, tyrosinase inhibitors, anti-inflammatory agents, skin lightening agents, and mixtures thereof. Many skin care actives may provide more than one benefit, or operate via more than one mode of action; therefore, classifications herein are made for the sake of convenience and are not intended to limit the active to that particular application or applications listed.

### 1. Vitamins

The compositions of the present invention may comprise from about 0.0001% to about 50%, alternatively from about 0.001% to about 10%, alternatively from about 0.01% to about 5%, and alternatively from about 0.1% to about 1%, of one or more vitamins. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

### 2. Peptides and Peptide Derivatives

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins (Ridulisse C™, from Silab, France), carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®).

The compositions may comprise from about 1×10⁻⁷% to about 20%, alternatively from about 1×10⁻⁶% to about 10%, and alternatively from about 1×10⁻⁵% to about 5% of the peptide.

### 3. Sugar Amines

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

### 4. Sunscreens

The compositions of the subject invention may comprise one or more sunscreen actives (or sunscreen agents) and/or ultraviolet light absorbers. Herein, “sunscreen active” includes both sunscreen agents and physical sunblocks. Sunscreen actives and ultraviolet light absorbers may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreen actives include benzophenone-3, benzylidene camphor sulfonic acid, cinoxate, dea-methoxycinnamate, dibenzoxazoyl naphthalene, di-t-butyl hydroxy-benzylidene camphor, diethylhexyl butamido triazone, ethylhexyl methoxy-cinnamate, ethylhexyl methoxydibenzoyl-methane, ethylhexyl salicylate, ethylhexyl triazone, ethyl methoxycinnamate, menthyl anthranilate, 4-methylbenzylidene camphor, octocrylene, octrizole, phenylbenzimidazole sulfonic acid, titanium dioxide, zinc oxide, and mixtures thereof. In one embodiment, the composition comprises from about 1% to about 20%, and alternatively from about 2% to about 10% by weight of the composition, of the sunscreen active and/of ultraviolet light absorber. Exact amounts will vary depending upon the chosen sunscreen active and/or ultraviolet light absorber and the desired Sun Protection Factor (SPF), and are within the knowledge and judgment of one of skill in the art.

### 5. Oil Control Agents

The compositions of the present invention may comprise one or more compounds useful for regulating the production of skin oil, or sebum, and for improving the appearance of oily skin. Examples of suitable oil control agents include salicylic acid, dehydroacetic acid, benzoyl peroxide, resorcinol, sulfur, erythromycin, zinc, vitamin B3 compounds (for example, niacinamide or tocopheryl nicotinate), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

### 6. Flavonoids

The compositions of the present invention may comprise a flavonoid. The flavonoid can be synthetic materials or obtained as extracts from natural sources, which also further may be derivatized. Examples of classes of suitable flavonoids are disclosed in U.S. Pat. No. 6,235,773, issued to Bissett, and include, but are not limited to, unsubstituted flavanones, methoxy flavanones, unsubstituted chalcones, and mixtures thereof. In one embodiment, the flavonoids are unsubstituted flavanones, unsubstituted chalcone (especially the trans-isomer), their glucosyl derivatives, and mixtures thereof. Other examples of suitable flavonoids include flavanones such as hesperidin and glucosyl hesperidin, isoflavones such as soy isoflavones, including but not limited to genistein, daidzein, quercetin, and equol, their glucosyl derivatives, 2′,4-dihydroxy chalcone, and mixtures thereof.

The compositions of the present invention may comprise from about 0.01% to about 20%, alternatively from about 0.1% to about 10%, and alternatively from about 0.5% to about 5% of flavonoids.

### 7. Hair Growth Regulators

The compositions of the present invention may comprise compounds useful for regulating hair growth. Suitable hair growth regulators include, but are not limited to, hexamidine, butylated hydroxytoluene (BHT), hexanediol, panthenol and pantothenic acid derivates, their isomers, salts and derivatives, and mixtures thereof. The compositions of the present invention may comprise from about 0.0001% to about 20%, alternatively from about 0.001% to about 10%, alternatively from about 0.01% to about 5%, and alternatively from about 0.1% to about 2% of hair growth regulators.

### 8. Skin Lightening Agents

The present compositions may comprise from about 0.1% to about 10%, and alternatively from about 0.2% to about 5%, of a skin lightening agent. The skin lightening agent may improve the appearance of the skin and/or reduce hyperpigmentation. Useful whitening agents useful herein include azelaic acid, butyl hydroxy anisole, gallic acid, hydroquinoine, kojic acid, arbutin and deoxy-arbutin, mulberry extract, undecylenoyl phenylalanine, octadecenedioic acid, octadecenedioic acid, ascorbic acid and derivatives of any of the foregoing, and mixtures thereof.

### 9. Other Skin Care Actives

The compositions of the present invention further may comprise non-vitamin antioxidants and radical scavengers, minerals, preservatives, phytosterols and/or plant hormones, protease inhibitors, tyrosinase inhibitors, and anti-inflammatory agents.

Suitable non-vitamin antioxidants and radical scavengers include, but are not limited to, BHT (butylated hydroxy toluene), butylated hydroxy benzoic acids, L-ergothioneine (available as THIOTANE™), tetrahydrocurcumin, cetyl pyridinium chloride, diethylhexyl syrinylidene malonate (available as OXYNEX™), 6-hydroxy-2,5,7,8-tetramethylchroman-2-carboxylic acid (available as Trolox™), hexadec-8-ene-1,16-dicarboxylic acid (octadecene dioic acid; available as ARLATONE™ Dioic DCA from Uniqema), ubiquinone (co-enzyme Q10), tea extracts including green tea extract, yeast extracts or yeast culture fluid (e.g., Pitera™), gallic acid, uric acid, sorbic acid, lipoic acid, amines (e.g., N,N-diethylhydroxylamine, amino-guanidine), sulfhydryl compounds including glutathione, dihydroxy fumaric acid, lycine pidolate, arginine pilolate, nordihydroguaiaretic acid, curcumin, lysine, methionine, proline, superoxide dismutase, silymarin, grape skin/seed extracts, melanin, rosemary extracts, salts and derivatives of any of the foregoing, and combinations thereof.

Suitable minerals include zinc, manganese, magnesium, copper, iron, selenium and other mineral supplements. “Mineral” is understood to include minerals in various oxidation states, mineral complexes, salts, derivatives, and combinations thereof.

Suitable examples of plant sterols (phytosterols) and/or plant hormones include, but are not limited to, sitosterol, stigmasterol, campesterol, brassicasterol, kinetin, zeatin, and derivatives and mixtures thereof.

Suitable protease inhibitors include, but are not limited to, hexamidine, vanillin acetate, menthyl anthranilate, soybean trypsin inhibitor, Bowman-Birk inhibitor, and mixtures thereof.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to nonsteroidal anti-inflammatory agents (NSAIDS), including but not limited to ibuprofen, naproxen, flufenamic acid, etofenamate, aspirin, mefenamic acid, meclofenamic acid, piroxicam and felbinac; glycyrrhizic acid (also known as glycyrrhizin, glycyrrhixinic acid, and glycyrrhetinic acid glycoside), glycyrrhetenic acid, other licorice extracts; candelilla wax, bisabolol (e.g., alpha bisabolol), manjistha (extracted from plants in the genus Rubia, particularly Rubia cordifolia), and guggal (extracted from plants in the genus Commiphora, particularly Commiphora mukul), kola extract, chamomile, red clover extract, and sea whip extract, derivatives of any of the foregoing, and mixtures thereof.

Other useful skin care actives include moisturizing and/or conditioning agents, such as glycerol, petrolatum, aloe vera, allantoin, bisabolol, dipotassium glycyrrhizinate, and urea; dehydroepiandrosterone (DHEA), its analogs and derivatives; exfoliating agents, including alpha- and beta-hydroxyacids, alpha-keto acids, glycolic acid and octanoyl salicylate; desquamation actives, including zwitterionic surfactants; antimicrobial agents; anti-cellulite agents, such as caffeine, theophylline, theobromine, and aminophylline; antidandruff agents such as piroctone olamine, 3,4,4′-trichlorocarbanilide (trichlosan), triclocarban and zinc pyrithione; dimethyl aminoethanol (DMAE); creatine; (sunless) tanning agents, such as dihydroxy acetone (DHA); chelators, for example, furildioxime and furilmonoxime; dialkanoyl hydroxyproline compounds; soy extracts, such as soybean milk, soybean paste, and miso salts; amino acids; topical anaesthetics, such as benzocaine, lidocaine, bupivacaine, chlorprocaine, dibucaine, etidocaine, mepivacaine, tetracaine, dyclonine, hexylcaine, procaine, cocaine, ketamine, pramoxine, phenol; salts and derivatives of any of the foregoing; and mixtures thereof.

### I. Other Ingredients

### Thickening Agents

The compositions of the present invention may comprise from about 0.1% to about 5%, alternatively from about 0.1% to about 4%, and alternatively from about 0.25% to about 3%, of a thickening agent. Nonlimiting classes of thickening agents include but not limited to carboxylic acid polymers, crosslinked polyacrylate polymers, polyacrylamide polymers, polysaccharides and gums.

### Other Particulate Materials

The composition of the present invention may comprise from about 0.001% to about 15% of an additional, non-colorant particulate material. The particulate material may be hydrophilic or hydrophobic, coated or uncoated, and may be present in the aqueous phase and/or the non-aqueous phase. In one embodiment, the additional particulate material is non-hydrophilically coated. In one embodiment, the additional particulate material is selected from the group consisting of titanium dioxide, zinc oxide, mica, boron nitride, polyethylene, and mixtures thereof.

### II. Methods of Use

The present invention describes a method of regulating and/or improving the condition of mammalian skin. The method comprises the step of topically applying to mammalian skin a personal care composition described herein. In one embodiment, the method may comprise the step of applying the composition described herein to insult-affected keratinous tissue, to regulate and/or improve the condition of such tissue, and/or to provide relief from the effects of the insult. When the aqueous phase visibly separates from the non-aqueous phase upon application to the keratinous tissue, the composition may signal an immediate, or acute, benefit to a consumer and may increase the penetration of water soluble skin care actives into the keratinous tissue. Thus in an alternative embodiment, the present invention describes a method of signaling an immediate benefit to a consumer, comprising the step of applying a composition of the present invention.

The composition may be applied to any keratinous tissue, including keratinous tissue in need of one or more benefits. Benefits include regulating and/or improving the condition of keratinous tissue, non-limiting examples of which include reducing the appearance of wrinkles, reducing the appearance of deep lines, reducing the appearance of fine lines, reducing the appearance of large pores, reducing the thickness of keratinous tissue, increasing the convolution of the dermal-epidermal border, increasing elasticity, reducing the appearance of cellulite, reducing the appearance of discoloration, reducing the appearance of hyperpigmentation, reducing the appearance of under-eye circles, reducing the appearance of sallowness, and combinations thereof. Alternatively, the benefit may include reducing wrinkles, reducing deep lines, reducing fine lines, reducing large pores, reducing cellulite, reducing hyperpigmentation, reducing undereye circles, reducing puffiness, and combinations thereof.

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices. In one embodiment, the composition is gently spread onto the skin to facilitate the separation of the aqueous phase from the oil-phase. When the aqueous phase has separated and coalesced into visibly enhanced droplets, the composition may be left as is on the keratinous tissue. Alternatively, the composition allowed to remain on the skin for 5 seconds, 10 seconds, 30 seconds, or 1 minute prior to being rubbed into the keratinous tissue.

The amount of the composition applied, the frequency of application and the period of use will vary widely depending upon the level of components of a given composition and the level of regulation desired. For example, from about 0.1 mg composition/cm² to about 50 mg composition/cm², and alternatively about 2 mg composition/cm² of keratinous tissue may be applied. In one embodiment, the composition is applied prior to exposure of the skin to ultraviolet radiation, and alternatively at least once daily, where “daily” and “days” mean a 24-hour period. The composition further may be applied as part of a treatment regimen, for example, once daily for 30 consecutive days, alternatively for 14 consecutive days, alternatively for 7 consecutive days and alternatively for 2 consecutive days.

The method may comprise the step of inducing a temperature change in the composition and/or in the keratinous tissue either simultaneously or sequentially with the step of applying the composition. The method further may comprise additional steps which form part of a treatment or application regimen, including the steps of applying at least one additional composition, ingesting one or more dietary supplements, cleansing, etc.

### III. Kit

The present invention further provides a kit comprising at least one composition described herein. The kit may comprise an outer packaging unit, which in turn may comprise one or more inner packaging units, optionally suitable for a single application, and disposable after one use. The inner and outer packaging units may be of any type suitable for containing, presenting and/or reasonably protecting from damage the contents of the kit. The kit further may comprise a plurality of components, including but not limited to, one or more additional compositions, one or more orally ingestible dietary supplements, an implement, a delivery enhancement device, a temperature change element, instructions for use of the implement and/or device, instructions for complying with suitable application regimens, instructions for targeted delivery, a substrate, and combinations thereof.

### EXAMPLES 1-6

The following are non-limiting examples of compositions that may be applied to keratinous tissue in accordance with the methods described herein. Examples 1 and 2 are comparative examples and examples 3-6 exhibit visibly enhanced water release, as described herein.

| | Example | | | | | |
|---|---|---|---|---|---|---|
| | 1 | 2 | 3 | 4 | 5 | 6 |
| PHASE A | | | | | | |
| DC-9040 *1 | 13.5 | 3.00 | 3.00 | 3.00 | 15.00 | 15.00 |
| Dimethicone | | 4.00 | 4.00 | 4.00 | 4.00 | 4.00 |
| Polymethylsilsesquioxane *2 | 7.5 | 4.00 | 4.00 | 4.00 | 4.00 | 4.00 |
| Cyclomethicone | 23.5 | 3.00 | 7.00 | 7.00 | 6.00 | 6.00 |
| KSG-210 *3 | 2.5 | 2.75 | 2.75 | 2.75 | 2.75 | 2.75 |
| DC-2503 Cosmetic Wax *4 | | | | | | |
| Abil EM97 *5 | 0.50 | | | | | |
| KF 6017 *6 | 0.40 | | | | | |
| Cetyl Ricinoleate | 0.25 | | | | | |
| (Titanium Dioxide coated mica) | | | | | | |
| coated with 2% Triethoxycaprylsilane *7 | | | | | | |
| Fragrance | 0.10 | 0.10 | 0.10 | 0.10 | 0.10 | 0.10 |
| PHASE B | | | | | | |
| Glycerin | 7.00 | 10.00 | 10.00 | 10.00 | 10.00 | 10.00 |
| Panthenol | 1.00 | 0.5 | 0.5 | 0.5 | 0.5 | 0.5 |
| Pentylene Glycol | | 3.00 | 3.00 | 3.00 | 3.00 | 3.00 |
| Propylene Glycol | 1.00 | | | | | |
| Butylene Glycol | 1.00 | | | | | |
| Tocopherol Acetate | 0.50 | | | | | |
| Citric Acid | | | 0.03 | 0.03 | 0.03 | 0.03 |
| Sodium Citrate | | | 0.20 | 0.20 | 0.20 | 0.20 |
| Sodium Benzoate | | | 0.05 | 0.05 | 0.07 | 0.07 |
| Hexamidine Diisethionate *8 | | 0.10 | 0.10 | 0.10 | 0.10 | 0.10 |
| Niacinamide *9 | 1.00 | 5.00 | 5.00 | 5.00 | 5.00 | 5.00 |
| Methylparaben | 0.10 | 0.20 | 0.20 | 0.20 | 0.20 | 0.20 |
| Ethylparaben | 0.10 | 0.05 | 0.05 | 0.05 | 0.05 | 0.05 |
| Benzyl Alcohol | 0.50 | | | | | |
| Propyl Paraben | 0.10 | | | | | |
| Disodium EDTA | 0.10 | | | | | |
| Sodium Chloride | | 0.50 | 0.50 | 0.50 | 0.50 | 0.50 |
| Titanium Dioxide Dispersion*10 | | | 0.20 | 2.00 | | |
| FD&C Blue Dye #1 | | | | | 0.0005 | 0.001 |
| (Titanium Dioxide coated mica) | | | | | | |
| coated with 2% PEG-8 Methyl Ether | | | | | | |
| Triethoxysilane *11 | | | | | | |
| Iron Oxide (C.I. 77491) coated with | | | | | | |
| 2% PEG-8 Methyl Ether | | | | | | |
| Triethoxysilane *12 | | | | | | |
| Water | q.s to | q.s to 100 | q.s to 100 | q.s to 100 | q.s to 100 | q.s to |
| | 100 | | | | | 100 |
| PERFORMANCE | | | | | | |
| Water Release | No | Yes | Yes | Yes | Yes | Yes |
| Enhanced Visible Water | No | No | Yes | Yes | Yes | Yes |

*1. 12.5% Dimethicone Crosspolymer in Cyclopentasiloxane. Available from Dow Corning
*2. E.g.Tospearl 145A or Tospearl 2000. Available from GE Toshiba Silicone
*3. 25% Dimethicone PEG-10/15 Crosspolymer in Dimethicone. Available from Shin-Etsu
*4. Stearyl Dimethicone. Available from Dow Corning.
*5. Bis-PEG/PPG-14/14 Dimethicone. Available from Degussa
*6. PEG-10 Dimethicone. Available from Shin-Etsu.
*7. KTZ Fine White 11S2 from Kobo Products, Inc
*8. Hexamidine diisethionate, availabile from Laboratoires Serobiologiques.
*9. Additionally or alternatively, the composition may comprise one or more other skin care actives, their salts and derivatives, as disclosed herein, in amounts also disclosed herein as would be deemed suitable by one of skill in the art.
*1075% Titanium Dioxide and Water and Glycerin and Ammonium Polyacrylate from Kobo Products, Inc.
*11. KTZ InterFine Blue SW2 from Kobo Products, Inc.
*12. BGRO-SW2 From Kobo Products, Inc

For example 1, in a suitable container, combine the ingredients of Phase A and mix with a suitable mixer until homogenous. In a separate container, combine the ingredients of Phase B and mix until homogenous. Slowly add Phase B to Phase A while continuing to mix Phase A. Continue mixing until batch is uniform. Homogenize product with Ultra-Turrax homogenizer (IKA, Inc) or equivalent and pour product into suitable containers.

For examples 2-6, in a suitable container, combine the ingredients of Phase A. In a separate suitable container, combine the ingredients of Phase B. Mixing each phase using a suitable mixer (e.g., Anchor blade, propeller blade) until each is homogenous. Slowly add Phase B to Phase A while continuing to mix Phase A. Continue mixing until batch is uniform. Pour product into suitable containers and store at room temperature.

### EXAMPLES 7-11

The following are non-limiting examples of compositions that may be applied to keratinous tissue in accordance with the methods described herein. Examples 7-11 exhibit visibly enhanced water release, as described herein.

| | Example | | | | |
|---|---|---|---|---|---|
| | 7 | 8 | 9 | 10 | 11 |
| PHASE A | | | | | |
| DC-9040 *1 | 3.00 | 3.00 | 3.00 | 3.00 | 3.00 |
| Dimethicone | 4.00 | 4.00 | 4.00 | 4.00 | 4.00 |
| Polymethylsilsesquioxane *2 | 4.00 | 4.00 | 4.00 | 4.00 | 4.00 |
| Cyclomethicone | 3.00 | 3.00 | 3.00 | 0.45 | 0.45 |
| KSG-210 *3 | 2.75 | 2.75 | 2.75 | 4.40 | 4.40 |
| DC-2503 Cosmetic Wax *4 | | | | 10.00 | 10.00 |
| Abil EM97 *5 | | | | | |
| KF 6017 *6 | | | | | |
| Cetyl Ricinoleate | | | | | |
| (Titanium Dioxide coated mica) coated | | | | 0.50 | |
| with 2% Triethoxycaprylsilane *7 | | | | | |
| Fragrance | 0.10 | 0.10 | 0.10 | 0.10 | 0.10 |
| PHASE B | | | | | |
| Glycerin | 10.00 | 10.00 | 10.00 | 10.00 | 10.00 |
| Panthenol | 0.5 | 0.5 | 0.5 | 0.5 | 0.5 |
| Pentylene Glycol | 3.00 | 3.00 | 3.00 | 3.00 | 3.00 |
| Propylene Glycol | | | | | |
| Butylene Glycol | | | | | |
| Tocopherol Acetate | | | | | |
| Citric Acid | | | | 0.03 | 0.03 |
| Sodium Citrate | | | | 0.20 | 0.20 |
| Sodium Benzoate | | | | 0.05 | 0.05 |
| Hexamidine Diisethionate *8 | 0.10 | 0.10 | 0.10 | 0.10 | 0.10 |
| Niacinamide *9 | 5.00 | 5.00 | 5.00 | 5.00 | 5.00 |
| Methylparaben | 0.20 | 0.20 | 0.20 | 0.20 | 0.20 |
| Ethylparaben | 0.05 | 0.05 | 0.05 | 0.05 | 0.05 |
| Benzyl Alcohol | | | | | |
| Propyl Paraben | | | | | |
| Disodium EDTA | | | | | |
| Sodium Chloride | 0.50 | 0.50 | 0.50 | 0.50 | 0.50 |
| Titanium Dioxide Dispersion*10 | | | | | |
| FD&C Blue Dye #1 | | | | | |
| (Titanium Dioxide coated mica) coated | | 1.00 | 2.00 | 1.00 | 1.00 |
| with 2% PEG-8 Methyl Ether | | | | | |
| Triethoxysilane *11 | | | | | |
| Iron Oxide (C.I. 77491) coated with 2% | 0.20 | | | | |
| PEG-8 Methyl Ether Triethoxysilane *12 | | | | | |
| Water | q.s to 100 | q.s to 100 | q.s to 100 | q.s to 100 | q.s to 100 |
| PERFORMANCE | | | | | |
| Water Release | Yes | Yes | Yes | Yes | Yes |
| Enhanced Visible Water | Yes | Yes | Yes | Yes | Yes |

*1. 12.5% Dimethicone Crosspolymer in Cyclopentasiloxane. Available from Dow Corning
*2. E.g.Tospearl 145A or Tospearl 2000. Available from GE Toshiba Silicone
*3. 25% Dimethicone PEG-10/15 Crosspolymer in Dimethicone. Available from Shin-Etsu
*4. Stearyl Dimethicone. Available from Dow Corning.
*5. Bis-PEG/PPG-14/14 Dimethicone. Available from Degussa
*6. PEG-10 Dimethicone. Available from Shin-Etsu.
*7. KTZ Fine White 11S2 from Kobo Products, Inc
*8. Hexamidine diisethionate, availabile from Laboratoires Serobiologiques.
*9. Additionally or alternatively, the composition may comprise one or more other skin care actives, their salts and derivatives, as disclosed herein, in amounts also disclosed herein as would be deemed suitable by one of skill in the art.
*1075% Titanium Dioxide and Water and Glycerin and Ammonium Polyacrylate from Kobo Products, Inc.
*11. KTZ InterFine Blue SW2 from Kobo Products, Inc.
*12. BGRO-SW2 From Kobo Products, Inc

For examples 7-9, in a suitable container, combine the ingredients of Phase A. In a separate suitable container, combine the ingredients of Phase B. Mixing each phase using a suitable mixer (e.g., Anchor blade, propeller blade) until each is homogenous. Slowly add Phase B to Phase A while continuing to mix Phase A. Continue mixing until batch is uniform. Pour product into suitable containers and store at room temperature.

For examples 10-11, combine the ingredients of Phase A in a suitable container. In a separate suitable container, combine the ingredients of Phase B. Heat each phase to 75 C-80° C. while mixing each phase using a suitable mixer (e.g., Anchor blade, propeller blade) until each reaches temperature and is homogenous. Slowly add Phase B to Phase A while continuing to mix Phase A. Continue mixing until batch is uniform. Pour product into suitable containers at 75-80° C. Store containers at room temperature without disturbing for at least 12 hours.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stable personal care composition comprising:
a) from about 0.1% to about 15% of a non-emulsifying crosslinked siloxane elastomer;
b) from about 0.1% to about 15% of an emulsifying crosslinked siloxane elastomer;
c) from about 1% to about 40% of an elastomer solvent;
d) less than 0.1% of a solidifying agent;
e) an aqueous phase comprising from about 0.0001% to about 10%, by weight of the composition, of a hydrophilic colorant;
whereupon application of shear force to the composition a visibly enhanced aqueous phase is visibly separated from the non-aqueous phase.

2. The composition of claim 1, wherein the hydrophilic colorant is selected from the group consisting of dyes, lakes, toners, inorganic pigments, organic pigments, particulate materials, and mixtures thereof.

3. The composition of claim 1, wherein the hydrophilic colorant is selected from the group consisting of bismuth oxychloride, iron oxide, mica, barium, interference pigments, sulfate-treated mica, titanium dioxide, zinc oxide, zirconium oxide, silica, nylon, polyethylene, talc, styrene, polypropylene, ethylene/acrylic acid copolymer, sericite, aluminum oxide, silicone resin, barium sulfate, calcium carbonate, cellulose acetate, polymethyl methacrylate, and mixtures thereof.

4. The composition of claim 1, wherein the hydrophilic colorant is a coated particulate comprising a core material and a coating material.

5. The composition of claim 4, wherein the core material is selected from the group consisting of mica, iron oxide, titanium dioxide, boron nitride, interference pigments, and mixtures thereof.

6. The composition of claim 5 wherein the core material is selected from the group consisting of mica, interference pigments, and mixtures thereof.

7. The composition of claim 4, wherein the coating material is hydrophilic.

8. The composition of claim 7, wherein the coating material is selected from the group consisting of water-soluble amino acids, collagen, lecithin, polyacrylate/lecithin, chitosan, and mixtures thereof.

9. The composition of claim 7, wherein the coating material comprises an anchoring group and a stabilizing group.

10. The composition of claim 9, wherein the anchoring group is a silanol and the stabilizing group is a polar moiety.

11. The composition of claim 10, wherein the polar moiety is selected from the group consisting of polyethyleneoxide, polyethylene glycols, polypropylene glycol, polyethylene glycol alkyl, alkyl glycols, alkyl glycol ether, polyethylene glycol esters, polyalkylene oxide, polypropyleneoxides, polyglycerides and mixtures thereof.

12. The composition of claim 10, wherein the anchoring group is covalently bonded to the core material.

13. The composition of claim 9, wherein the anchoring group is a selected from the group consisting of carboxyls, carboxylic acid esters, hydroxyls, sulfonates, sulfates, phosphates, phosphonates, nitros, carbohydrates, ammonium salts, phosphate esters, carbonyls, aminos, amides, imides, aliphatic hydrocarbons, aromatic hydrocarbons, heterocyclic groups, polypropyleneoxides, silicones, fluorocarbons, polyesters, urethanes and mixtures thereof, and wherein the stabilizing group is selected from the group consisting of polyethyleneoxide, polyethylene glycols, polypropylene glycol, polyethylene glycol alkyl, alkyl glycols, alkyl glycol ether, polyethylene glycol esters, polyalkylene oxide, polypropyleneoxides, polyglycerides, carboxyl, carboxylic acid esters, hydroxyl, sulfonate, sulfate, phosphate, phosphonate, nitro, carbohydrate, ammonium salts, phosphate esters, carbonyl, amino, amide, imide, aliphatic hydrocarbons, aromatic hydrocarbons, heterocyclic groups, polypropyleneoxides, silicones, fluorocarbons, polyesters and mixtures thereof.

14. The composition of claim 4 wherein the coated particulate has a surface energy comprising a polar component and a dispersive component, wherein the polar component is greater than 20 mJ/m² and the dispersive component is greater than 15 mJ/m².

15. The composition of claim 1, further comprising from about 0.001% to about 5% of an additional emulsifier.

16. The composition of claim 1, further comprising from about 0.001% to about 15% of a non-hydrophilic particulate material.

17. The composition of claim 1, further comprising at least one additional skin care active.

18. The personal care composition of claim 17, wherein said additional skin care active is selected from the group consisting of vitamin B compounds, vitamin C compounds, vitamin E compounds, peptides, sugar amines, oil control agents, skin lightening agents, and combinations thereof.

19. The personal care composition of claim 17, wherein said skin care active is selected from the group consisting of niacinamide, palmitoyl-lysine-threonine, palmitoyl-lysine-threonine-threonine-lysine-serine, N-acetyl-D-glucosamine, salicylic acid, dehydroacetic acid, sodium dehydroacetate, hexamidine compounds, and combinations thereof.

20. A personal care composition comprising:
a. from about 0.1% to about 15% of an emulsifying silicone elastomer;
b. from about 0.1% to about 40% of at least one solidifying agent;
c. from about 1% to about 75% of an aqueous phase;
d. from about 0.1% to about 74% water;
e. from about 0.1% to about 10% of a hydrophilic coated particulate comprising a core material and a coating material;
wherein the composition is in the form of a water-in-oil emulsion, has a first hardness of from about 2 g to about 45 g at a first temperature of 21° C., and a second hardness at a second temperature of 33° C., wherein the second hardness is 65% or less of the first hardness; and whereupon application of shear force to the composition a visibly enhanced aqueous phase is visibly separated from the non-aqueous phase.

21. The composition of claim 20, wherein the coating material is selected from the group consisting of water-soluble amino acids, collagen, lecithin, polyacrylate/lecithin, chitosan, and mixtures thereof.

22. The composition of claim 20, wherein the coating material comprises an anchoring group and a stabilizing group.

23. The composition of claim 22, wherein the anchoring group is a silanol and the stabilizing group is a polar moiety.

24. The composition of claim 23, wherein the polar moiety is selected from the group consisting of polyethyleneoxide, polyethylene glycols, polypropylene glycol, polyethylene glycol alkyl, alkyl glycols, alkyl glycol ether, polyethylene glycol esters, polyalkylene oxide, polypropyleneoxides, polyglycerides and mixtures thereof.

25. The composition of claim 22, wherein the anchoring group is covalently bonded to the core material.

26. The composition of claim 22, wherein the anchoring group is a selected from the group consisting of carboxyls, carboxylic acid esters, hydroxyls, sulfonates, sulfates, phosphates, phosphonates, nitros, carbohydrates, ammonium salts, phosphate esters, carbonyls, aminos, amides, imides, aliphatic hydrocarbons, aromatic hydrocarbons, heterocyclic groups, polypropyleneoxides, silicones, fluorocarbons, polyesters, urethanes and mixtures thereof, and wherein the stabilizing group is selected from the group consisting of polyethyleneoxide, polyethylene glycols, polypropylene glycol, polyethylene glycol alkyl, alkyl glycols, alkyl glycol ether, polyethylene glycol esters, polyalkylene oxide, polypropyleneoxides, polyglycerides, carboxyl, carboxylic acid esters, hydroxyl, sulfonate, sulfate, phosphate, phosphonate, nitro, carbohydrate, ammonium salts, phosphate esters, carbonyl, amino, amide, imide, aliphatic hydrocarbons, aromatic hydrocarbons, heterocyclic groups, polypropyleneoxides, silicones, fluorocarbons, polyesters and mixtures thereof.

27. The composition of claim 20 wherein the coating material has a surface energy comprising a polar component and a dispersive component, wherein the polar component is greater than 20 mJ/m² and the dispersive component is greater than 15 mJ/m².

28. The composition of claim 20, wherein the core material is selected from the group consisting of mica, iron oxide, titanium dioxide, boron nitride, interference pigments, and mixtures thereof.

29. The composition of claim 20, wherein the core material is selected from the group consisting of mica, interference pigments, and mixtures thereof.

30. The composition of claim 20, wherein the hydrophilic coated particulate has an average size of from about 0.1 microns to about 100 microns.

31. The composition of claim 20, further comprising from about 0.1% to about 15% of a non-emulsifying silicone elastomer.

32. The composition of claim 20, wherein the solidifying agent is a wax.

33. The composition of claim 32, wherein the wax is selected from the group consisting of a silicone wax, a polyethylene wax, a synthetic wax, and mixtures thereof.

34. The composition of claim 32, wherein the wax is selected from the group consisting of C16 to C28 alkyl dimethicone waxes, polythethylene waxes with a melting point of less than 120° C., synthetic waxes with a melting point of less than 120° C., and mixtures thereof.

35. The composition of claim 20, wherein the solidifying agent comprises from about 0.1% to about 20% of a first wax having a first melting point of from about 21° C. to about 40° C., and from about 0.1% to about 20% of a second wax having a second melting point of from about 40° C. to about 120° C.

36. The composition of claim 20, wherein the first hardness at a first temperature of 21° C. is from about 2 g to about 45 g and the second hardness at a second temperature of 33° C. is about 0.1 g to about 30 g.

37. The composition of claim 20, further comprising from about 0.001% to about 15% of a non-hydrophilic coated particulate material.

38. The composition of claim 20, further comprising at least one additional skin care active.

39. The personal care composition of claim 38, wherein said additional skin care active is selected from the group consisting of vitamin B compounds, vitamin C compounds, vitamin E compounds, peptides, sugar amines, oil control agents, skin lightening agents, and combinations thereof.

40. The personal care composition of claim 38, wherein said skin care active is selected from the group consisting of niacinamide, palmitoyl-lysine-threonine, palmitoyl-lysine-threonine-threonine-lysine-serine, N-acetyl-D-glucosamine, salicylic acid, dehydroacetic acid, sodium dehydroacetate, hexamidine compounds, and combinations thereof.

41. A method of providing a benefit to mammalian keratinous tissue, comprising the step of topically applying to mammalian keratinous tissue a personal care composition comprising:
a) from about 0.1% to about 15% of a non-emulsifying crosslinked siloxane elastomer;
b) from about 0.1% to about 15% of an emulsifying crosslinked siloxane elastomer;
c) from about 1% to about 40% of an elastomer solvent;
d) less than 0.1% of a solidifying agent;
e) an aqueous phase comprising from about 0.0001% to about 10%, by weight of the composition, of a hydrophilic colorant;
whereupon application of shear force to the composition a visibly enhanced aqueous phase is visibly separated from the non-aqueous phase.

42. The method of claim 41, wherein the mammalian keratinous tissue is insult-affected mammalian skin.

43. A method of providing a benefit to mammalian keratinous tissue, comprising the step of topically applying to mammalian keratinous tissue a personal care composition comprising:
a. from about 0.1% to about 15% of an emulsifying silicone elastomer;
b. from about 0.1% to about 40% of at least one solidifying agent;
c. from about 1% to about 75% of an aqueous phase;
d. from about 0.1% to about 74% water;
e. from about 0.1% to about 10% of a hydrophilic coated particulate comprising a core material and a coating material;
wherein the composition is in the form of a water-in-oil emulsion, has a first hardness of from about 2 g to about 45 g at a first temperature of 21° C., and a second hardness at a second temperature of 33° C., wherein the second hardness is 65% or less of the first hardness; and whereupon application of shear force to the composition a visibly enhanced aqueous phase is visibly separated from the non-aqueous phase.

44. The method of claim 43, wherein the mammalian keratinous tissue is insult-affected mammalian skin.

45. A kit comprising:
a) a personal care composition comprising:
i. from about 0.1% to about 15% of a non-emulsifying crosslinked siloxane elastomer;
ii. from about 0.1% to about 15% of an emulsifying crosslinked siloxane elastomer;
iii. from about 1% to about 40% of an elastomer solvent;
iv. less than 0.1% of a solidifying agent;
v. an aqueous phase comprising from about 0.0001% to about 10%, by weight of the composition, of a hydrophilic colorant;
whereupon application of shear force to the composition a visibly enhanced aqueous phase is visibly separated from the non-aqueous phase;
b) at least one additional component selected from the group consisting of at least one additional composition, at least one orally ingestible dietary supplement, an implement, a delivery enhancement device, a temperature change element, instructions for complying with suitable application regimens, and combinations thereof; and
c) instructions for complying with a regimen to provide a benefit to keratinous tissue.

46. A kit comprising:
a) a personal care composition comprising:
a. from about 0.1% to about 15% of an emulsifying silicone elastomer;
b. from about 0.1% to about 40% of at least one solidifying agent;
c. from about 1% to about 75% of an aqueous phase;
d. from about 0.1% to about 74% water;
e. from about 0.1% to about 1% of a hydrophilic coated particulate comprising a core material and a coating material;
wherein the composition is in the form of a water-in-oil emulsion, has a first hardness of from about 2 g to about 45 g at a first temperature of 21° C., and a second hardness at a second temperature of 33° C., wherein the second hardness is 65% or less of the first hardness; and whereupon application of shear force to the composition a visibly enhanced aqueous phase is visibly separated from the non-aqueous phase;
b) at least one additional component selected from the group consisting of at least one additional composition, at least one orally ingestible dietary supplement, an implement, a delivery enhancement device, a temperature change element, instructions for complying with suitable application regimens, and combinations thereof; and
c) instructions for complying with a regimen to provide a benefit to keratinous tissue.
